(19) United States
(12) Patent Application Publication
(10) Pub. No.: US 2020/0093195 A1
(43) Pub. Date: Mar. 26, 2020

(54) SLEEPING GARMENT ASSEMBLY

(71) Applicant: Justin Whitaker, Antioch, TN (US)

(72) Inventor: Justin Whitaker, Antioch, TN (US)

(21) Appl. No.: 16/136,390

(22) Filed: Sep. 20, 2018

(51) Int. Cl. A41D 1/04; A41D 27/00; A41D 27/20; A47G 9/10; A61F 9/04

(52) U.S. Cl. CPC A41D 1/04; A41D 27/00; A41D 27/20; A41D 2300/322; A47G 9/1045; A61F 9/045; A41D 2200/20; A41D 27/201

(57) ABSTRACT

A sleeping garment assembly for facilitating a user to sleep upright includes a garment that is worn on a user's upper body. The garment has a hood that is positionable over the user's head and the hood is hollow. A pillow is removably positioned in the hood to cushion the user's head for sleeping.

### CROSS-REFERENCE TO RELATED APPLICATIONS

[symbol]

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

[symbol]

### (2) Description of Related Art Including Information Disclosed Under 37 CFR 1.97 and 1.98

The disclosure and prior art relates to garment devices and more particularly pertains to a new garment device for facilitating a user to sleep upright.

### BRIEF SUMMARY OF THE INVENTION

An embodiment of the disclosure meets the needs presented above by generally comprising a garment that is worn on a user's upper body. The garment has a hood that is positionable over the user's head and the hood is hollow. A pillow is removably positioned in the hood to cushion the user's head for sleeping.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an exploded perspective view of a sleeping garment assembly according to an embodiment of the disclosure.

FIG. 2 is a cross sectional view taken along line 2-2 of FIG. 1 of an embodiment of the disclosure.

FIG. 3 is a perspective in-use view of an embodiment of the disclosure.

### DETAILED DESCRIPTION OF THE INVENTION

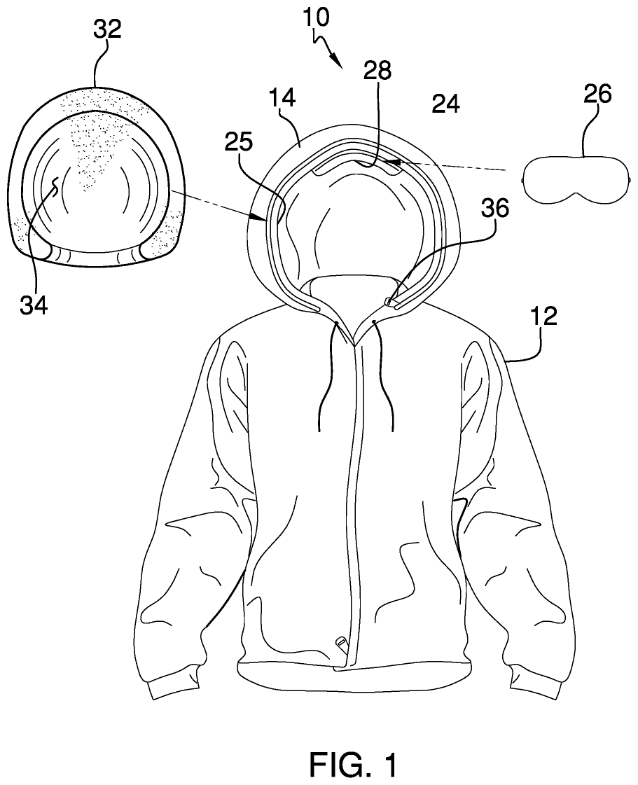

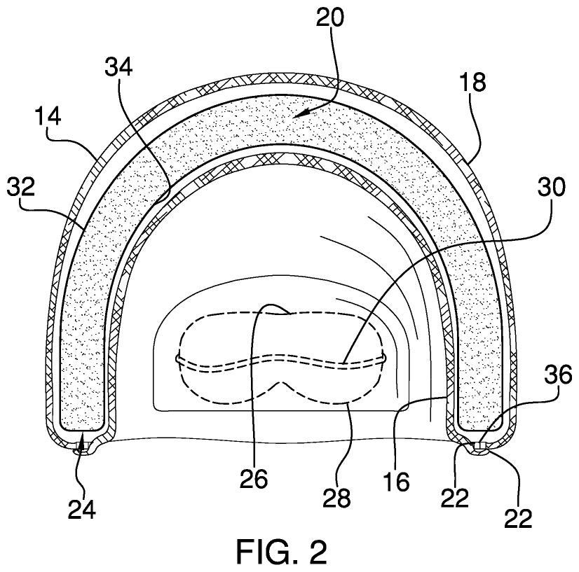

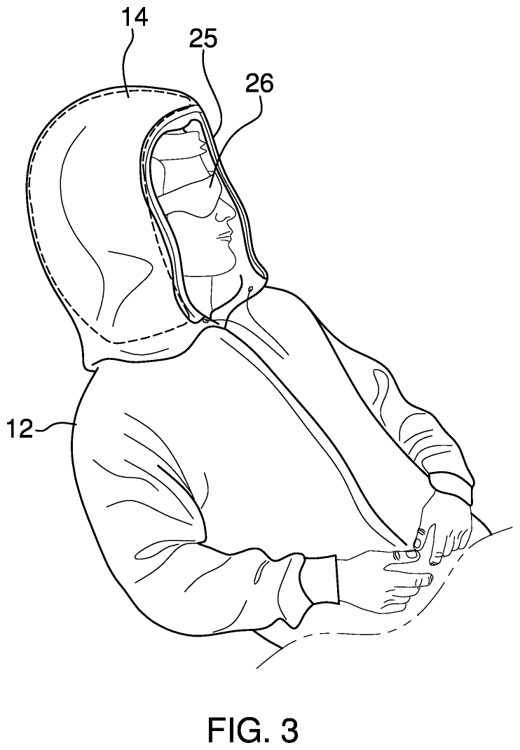

With reference now to the drawings, and in particular to FIGS. 1 through 3 thereof, a new garment device embodying the principles and concepts of an embodiment of the disclosure and generally designated by the reference numeral 10 will be described.

As best illustrated in FIGS. 1 through 3, the sleeping garment assembly 10 generally comprises a garment 12 that is worn on a user's upper body. The garment 12 has a hood 14 that is positionable over the user's head and the hood 14 is hollow. The garment 12 has a bodice and a pair of sleeves, and the hood 14 has an inside wall 16 and an outside wall 18. The inside wall 16 is spaced from the outside wall 18 to define a space 20 within the hood 14 such that the hood 14 is hollow. Each of the inside 16 and outside 18 walls has a first edge 22. The first edge 22 of the inside wall 16 is aligned with and is spaced from the first edge 22 of the outside wall 18 to define an opening 24 to access the space 20. The opening 24 extends around a front perimeter 25 of the hood 14.

A mask 26 is provided and the mask 26 worn over the user's eyes when the user sleeps. The mask 26 may be a sleeping mask of any conventional design. A pocket 28 is coupled to the inside wall 16 of the hood 14 such that the pocket 28 is accessible. The pocket 28 has an upper edge 30 and the upper edge 30 is open for receiving the mask 26 for storage. The pocket is spaced from the front perimeter 25 such that the mask is accessible when the hood is worn on the user's head.

A pillow 32 is removably positioned in the hood 14 to cushion the user's head for sleeping. In this way the user can sleep while sitting up during air travel, car travel or any other situation that involved the user sitting upright for extended periods of time. The pillow 32 has a first surface 34 and the first surface 34 is concavely arcuate such that the pillow 32 has a bowl shape. In this way the pillow 32 can conform to the users head. The pillow 32 is extendable through the opening 24 to position the pillow 32 in the space 20 and the pillow 32 is comprised of a resiliently compressible material.

A closure 36 is coupled to the hood 14 and the closure 36 is aligned with the first edge 22 of each of inside and outside wall 18s. The closure 36 closes the opening 24 when the closure 36 is closed and the closure 36 facilitates the opening 24 to be opened when the closure 36 is opened. The closure 36 retains the pillow 32 in the space 20 when the closure 36 is closed. Moreover, the closure 36 may comprise a zipper, a pair of complementary mating members or any other type of releasable closure. Additionally, the closure 36 may be integrated into the hood 14 in a manner that inhibits the closure 36 from being visible to an observer.

In use, the pillow 32 is inserted through the opening 24 unit the pillow 32 is fully positioned within the hood 14. The closure 36 is manipulated to close the opening 24 and thereby retain the pillow 32 in the hood 14. In this way the pillow 32 can cushion the user's head while the user sleeps. Additionally, the mask 26 can be removed from the pouch and worn while the user sleeps. In this way the pillow 32 and the mask 26 increase the quality of the user's sleep in what would otherwise be an uncomfortable situation for sleeping.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sleeping garment assembly having an integrated pillow for cushioning a user's head during sleep, said assembly comprising:
a garment being worn on a user's upper body, said garment having a hood being positionable over the user's head, said hood being hollow; and
a pillow being removably positioned in said hood wherein said pillow is configured to cushion the user's head for sleeping.

2. The assembly according to claim 1, wherein said hood has an inside wall and an outside wall, said inside wall being spaced from said outside wall to define a space within said hood such that said hood is hollow, each of said inside and outside walls having a first edge, said first edge of said inside wall being aligned with and being spaced from said first edge of said outside wall to define an opening to access said space, said opening extending around a front perimeter of said hood.

3. The assembly according to claim 2, further comprising:
a mask being worn over the user's eyes when the user sleeps; and
a pocket being coupled to said inside wall of said hood such that said pocket is accessible, said pocket having an upper edge, said upper edge being open for receiving said mask for storage.

4. The assembly according to claim 2, wherein said pillow has a first surface, said first surface being concavely arcuate such that said pillow has a bowl shape wherein said pillow is configured to conform to the users head, said pillow being extendable through said opening to position said pillow in said space, said pillow being comprised of a resiliently compressible material.

5. The assembly according to claim 4, further comprising a closure being coupled to said hood, said closure being aligned with said first edge of each of inside and outside walls, said closure closing said opening when said closure is closed, said closure facilitating said opening to be opened when said closure is opened, said closure retaining said pillow in said space when said closure is closed.

6. A sleeping garment assembly having an integrated pillow for cushioning a user's head during sleep, said assembly comprising:
a garment being worn on a user's upper body, said garment having a hood being positionable over the user's head, said hood being hollow, said garment having a bodice and a pair of sleeves, said hood having an inside wall and an outside wall, said inside wall being spaced from said outside wall to define a space within said hood such that said hood is hollow, each of said inside and outside walls having a first edge, said first edge of said inside wall being aligned with and being spaced from said first edge of said outside wall to define an opening to access said space, said opening extending around a front perimeter of said hood;
a mask being worn over the user's eyes when the user sleeps;
a pocket being coupled to said inside wall of said hood such that said pocket is accessible, said pocket having an upper edge, said upper edge being open for receiving said mask for storage;
a pillow being removably positioned in said hood wherein said pillow is configured to cushion the user's head for sleeping, said pillow having a first surface, said first surface being concavely arcuate such that said pillow has a bowl shape wherein said pillow is configured to conform to the users head, said pillow being extendable through said opening to position said pillow in said space, said pillow being comprised of a resiliently compressible material; and
a closure being coupled to said hood, said closure being aligned with said first edge of each of inside and outside walls, said closure closing said opening when said closure is closed, said closure facilitating said opening to be opened when said closure is opened, said closure retaining said pillow in said space when said closure is closed.
